(19) United States
(12) Patent Application Publication
(10) Pub. No.: US 2007/0048323 A1
(43) Pub. Date: Mar. 1, 2007

(54) Antibody treatment of lipomatous tumors

(76) Inventor: J. Peter Rubin, Pittsburgh, PA (US)

Correspondence Address: Raymond A. Miller;Pepper Hamilton LLP, One Mellon Center, 50th Floor, 500 Grant Street, Pittsburgh, PA, 15219, US

(21) Appl. No.: 11/374,284

(22) Filed: Feb. 18, 2005

Related U.S. Application Data
(60) Provisional application No. 60/546,681, filed on Feb. 20, 2004.

(51) Int. Cl. A61K 39/395

(52) U.S. Cl. 424/155.1

(57) ABSTRACT

A method of treating lipomatous tumors in mammals such as humans or companion animals is described. The treatment of lipomatous tumors use antibodies derived from adipose tissue of a mammal donor. The methods of the present invention are effective against both benign and malignant lipomatous tumors. The antibodies of the present invention are used to decrease the size of lipomatous tumors, prevent the regrowth of removed tumors, and inhibit the proliferation of lipomatous cells.

### CROSS REFERENCE TO RELATED APPLICATION

This application claims priority to U.S. Provisional Patent Application No. 60/546,681, filed Feb. 20, 2004, titled “ANTIBODY TREATMENT OF LIPOMATOUS TUMORS, which is incorporated herein by reference.

### FIELD OF THE INVENTION

The present invention relates to a method of treating lipomatous and sarcomatous tumors in humans and companion animals.

### BACKGROUND OF THE INVENTION

Lipomatous tumors are very common in humans. They are derived from adipose cells and demonstrate neoplastic growth independent of dietary intake and behavior of the surrounding normal fat tissue. These lesions occur in almost every anatomic site of the body, but are primarily found in the subcutaneous tissue as a benign growth, with an incidence estimated as high as 1:1,000. The benign form of lipomatous tumors is referred to as lipoma, while the rare malignant variant is termed liposarcoma.

Lipomas are benign tumors composed of mature fat cells. They are the most common benign mesenchymal tumor. Lipomas are found in the subcutaneous tissues and, less commonly, in internal organs. They usually present with little difficulty in diagnosis or morbidity. Lipomas typically develop as discrete rubbery masses in the subcutaneous tissues of the trunk and proximal extremity. They usually are a few centimeters in size and can be removed by surgical excision or liposuction. Lipomas differ biochemically from normal fat by demonstrating increased levels of lipoprotein lipase and by the presence of a larger number of precursor cells. The incidence of malignancy in these lesions is rare.

One treatment for lipomas and liposarcomas alike is surgical excision. An example of such surgery includes liposuction using a small diameter cannula, which is usually a minimally invasive treatment. While liposuction involves a smaller skin incision than open excision, it is still a surgical procedure that requires anesthesia, special equipment, and a skilled practitioner.

The malignant form of lipomatous tumors, liposarcoma, is uncommon. The standard treatment of liposarcoma involves surgical excision often combined with radiation therapy. Local recurrence rates are high after surgical excision.

There is a need for less invasive treatment of lipomatous tumors. It has now been found that an injectable antibody therapy is useful in treating lipomatous tumors.

### SUMMARY OF THE INVENTION

The present invention relates to various methods of treating lipomatous tumors, both benign and malignant, in mammals. One embodiment of the present invention is a method of treating lipomatous tumors in mammals comprising administering a therapeutic amount of an antibody directly to tumor cells wherein the antibody is derived from adipose tissue of a mammal. Various method embodiments of the present invention may comprise the intralesional injection of antibody treatments.

Another embodiment of the present invention is a method of decreasing the number of lipomatous tumor cells comprising administering a therapeutic amount of an antibody directly to tumor cells of a subject.

Another embodiment of the present invention is a method of reducing the size of an lesion of lipomatous cells comprising administering a therapeutic amount of an antibody directly to the cells of a subject. Another embodiment of the present invention is a method of inhibiting growth of lipomatous tumor cells comprising administering a therapeutic amount of an antibody directly to tumor cells of a subject. Another embodiment of the present invention is a method of preventing the regrowth of a lesion of lipomatous cells comprising removing a lesion by surgical excision and administering a therapeutic amount of an antibody to tissue surrounding the removed lesion.

Another embodiment of the present invention is a composition for the treatment of lipomatous tumors comprising therapeutic antibodies directed against adipocytes.

Lipomatous cells and tumors are soft tissue neoplasms. Therefore, embodiments of the present invention are directed to any lipomatous cells and tumors, generally described as any neoplasm with cells containing adipose cells or cells histologically derived from adipose cells.

Aspects and applications of the present invention will become apparent to the skilled artisan upon consideration of the detailed description of the invention and the appended claims, which follow.

### DESCRIPTIONS OF THE DRAWINGS

Embodiments of the invention are illustrated by way of example and not limitation in the accompanying figure:

FIG. 1 is a diagram of the mechanism in which an antibody is directed against a fat cell membrane and binds to adipose derived tumor cells.

### DETAILED DESCRIPTION

For simplicity and illustrative purposes, the principles of the invention are described by referring mainly to an embodiment thereof. In the following description, numerous specific details are set forth in order to provide a thorough understanding of the invention. It will be apparent however, to one of ordinary skill in the art, that the invention may be practiced without limitation to these specific details. In other instances, well known methods and structures have not been described in detail so as not to unnecessarily obscure the invention.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference in their entirety. Nothing herein is to be construed as an admission that the invention is not entitled to antedate such disclosure by virtue of prior invention.

What has been described and illustrated herein are embodiments of the invention along with some of their variations. The terms, descriptions and figures used herein are set forth by way of illustration only and are not meant as limitations. Those skilled in the art will recognize that many variations are possible within the spirit and scope of the invention, which is intended to be defined by the following claims and their equivalents in which all terms are meant in their broadest reasonable sense unless otherwise indicated.

The present invention relates to various methods of treating lipomatous tumors, both benign and malignant, in mammals. As used in the present application the term “lipomatous tumors” or “lipomatous cells” generally refers to a broad category of lesions which include neoplams containing adipose cells or cells histologically derived from adipose cells. Therefore, the methods and compositions of the present invention will refer generally to “lipomatous tumors” or “lipomatous cells”, which is meant to include any one of this broad category of cells and/or neoplasms.

Various lipomatous tumors are described by N. de Saint Aubain Somerhausen, in his article “Lipomatous Tumors Of Soft Tissues”, Institut Jules Bordet, available at http://www.forpath.org/0001/partl.htm., herein incorporated by reference in its entirety. Other sources have described lipomatous cells. For example, the article of Reimann, et al., entitled, “Cytogenetic investigation of canine lipomas,” in Cancer Genetics & Cytogenetics. 111(2):172-4, 1999, herein incorporated by reference in its entirety, describes genes associated with tumor development. Additionally, the reference of Rydholm, et al., entitled, “Size, site and clinical incidence of lipoma. Factors in the differential diagnosis of lipoma and sarcoma”, in Acta Orthopaedica Scandinavica. 54(6):929-34, 1983, herein incorporated by reference in its entirety, describes methods of distinguishing between various lipomatous tissue.

A general description of several lipomatous tumors is provided. Lipomatous tumors are the most common soft tissue neoplasms. These lesions, which can occur at every age and at almost any anatomical location are part of the daily practice of every surgical pathologist. Generally, lipoma tumors are spindle cell and liposarcoma are round cell.

One approach to categorizing lipomatous cells are to group these lesions according to the amount of their fatty component: lesions composed predominantly of adipocytes, lesions accompanied by other connective tissue elements, and lipomatous tumors in whom the lipomatous differentiation is not always obvious. Histologic features which should be analyzed when examining a lipomatous lesion include the pattern of lobulation, the presence of other connective tissue elements, the regularity in size of adipocytes, the presence of atypical cells and lipoblasts.

Lipoblasts are not only characterized by the presence of lipid droplets scalloping their nuclei but should also show some nuclear enlargement and hyperchromatism, which differentiates them from atrophic adipocytes or foamy macrophages.

Lipomatous tumors are usually diagnosed on light-microscopic morphology alone. Immunohistochemistry is also of value, although staining for S100 protein may help to identify lipoblasts, and positivity for CD34 may support a diagnosis of spindle cell lipoma. Although presently it can hardly be performed in the general practice, the karyotype is another technique in the field of lipomatous tumors. The identification of chromosomal abnormalities in a large number of them have provided interesting insights into the relationships between various tumor types and some of these abnormalities are sufficiently (albeit not entirely) specific to play a diagnostic role in morphologically difficult cases.

Lipoma represents by far the most common mesenchymal neoplasm. Lipomas are rare during the first 2 in human decades and usually appears between 40 and 60 years. Most (but not all) lesions are superficial. They occur predominantly in the upper back or neck, abdomen, and proximal portions of the extremities and are multiple in 5 to 10% of cases.

Histologically, they are composed of mature fat cells showing only slight variations in size and shape. Nuclei are fairly uniform within lipoma. Intranuclear vacuoles are commonly found and should not be regarded as evidence for lipoblastic activity.

Secondary changes are not uncommon and may cause diagnostic problems. Traumatized lesions contain foci of fat necrosis, characterized by nests of foamy macrophages distributed in the intercellular spaces or around lipocytes. These macrophages must be differentiated from lipoblasts which have hyperchromatic, usually enlarged nuclei.

It is interesting to note that despite their close resemblance to normal adipose tissue, most lipomas are characterized by karyotypic abnormalities, usually involving chromosome 12.

Lipomas containing a significant fibrous component are sometimes called “fibrolipoma”. Small foci of myxoid changes are occasionally found. In rare cases, designated as “myxolipoma”, these changes may be extensive and their rich vascularity may become clearly apparent. The transition with classical areas as well as the absence of lipoblasts and of a plexiform vascular pattern allows the distinction from myxoid liposarcoma.

Foci of cartilaginous or osseous metaplasia are other occasionnal findings. Intramuscular lipoma affects middle-aged adults, with a predilection for the thigh and trunk. These tumors are usually poorly circumscribed and tend to extend between variably atrophic skeletal muscle fibers. These lesions may recur in up to 20% of cases if incompletely excised.

Lipomatosis is an uncommon condition characterized by an abnormal overgrowth of mature fatty tissue. Different forms have been described. Diffuse lipomatosis affects large portions of an extremity or trunk. Symmetric lipomatosis affects the neck and shoulders. Pelvic lipomatosis involves the perirectal and perivesical regions and may cause urinary obstruction. Steroid induced lipomatosis results primarily in a “buffalo hump” adipose deformity on the upper back and neck. HIV related lipodystrophy, believed to be caused by protease inhibitor medications, also involves abnormal collections of fat in the upper back and neck.

Pleomorphic lipoma is a benign tumor which forms with spindle-cell lipoma a spectrum of lesions with overlapping clinical, morphologic and cytogenetic characteristics.

Lipoblastoma is a rare benign tumor of infancy, affecting children principally before the age of 7 years. These lesions have a predilection for the limbs and are usually superficial. Deeper tumors, which tend to be larger and more infiltrative, are known as lipoblastomastosis.

Histologically, lipoblastoma is characterized by a prominent lobular architecture. Lobules, which are separated by fibrous septa, are composed of mature and immature fat cells, in variable proportions. Immature lesions, composed of primitive mesenchymal cells and lipoblasts, in a richly vascularized myxoid stroma, are virtually indistinguishable from myxoid liposarcoma.

In this differential diagnosis, the age of the patient is of greatest importance, as myxoid liposarcomas are exceedingly rare under the age of 10 years. A striking lobulation, with fibrous septa, and some maturation in the center of lobules (whereas in myxoid liposarcoma, maturation is seen in their periphery) also favor lipoblastoma. Cytogenetics may be helpful as lipoblastoma shows consistent rearrangements of chromosome 8.

Hibernoma is a rare tumor showing differentiation towards brown fat. It occurs in young adults, with a predilection for the scapular and interscapular region.

Atypical lipomatous tumor, otherwise known as well differentiated liposarcoma (“lipoma-like” variant) accounts for 40 to 45% of all liposarcomas and the lipoma-like form is by far the most common of its variants. Grossly, these tumors are usually well circumscribed, but tend to be larger and coarsely lobulated, with pale, firmer areas.

Histologically, lipoma-like liposarcoma is characterized by a variation in size of adipocytes, some of which showing atypical, hyperchromatic nuclei. Bizarre, often multinucleated stromal cells are often found in fibrous septa, or occasionally in vessel walls. Typical lipoblasts, which are often hard to find and may be absent, are not required to the diagnosis of atypical lipomatous tumor well differentiated liposarcoma.

Virtually all lesions, with or without lipoblasts are characterized cytogenetically by ring or long marker chromosomes derived from the q13-15 region of chromosome 12.

In the absence of a dedifferentiated component, well-differentiated liposarcoma does not metastasize and lesions that can be surgically excised (usually outside the retroperitoneum) are usually cured. For this reason, the term “atypical lipoma” may be used for well-differentiated tumors of the extremities. Others have expanded this definition to all tumors characterized by atypical lesions lacking lipoblasts, regardless of their location, or to all superficial lesions.

Tumors with admixed adipocytic and other connective tissue elements include angiolipoma. These rather common lesions differ from common lipomas by the fact that they occur in young adults, are often painful and are multiple in ⅔ of cases. There is a predilection for the forearm (⅔ of cases), arm and chest wall. Microscopically, they are defined by the presence of variable amounts of small vessels, some of which contain fibrinous microtrombi. Lesions occuring on the chest wall must be differentiated from well differentiated angiosarcoma of the breast.

Spindle cell/pleomorphic lipoma has a typical clinical setting: these lesions mainly affect males in their mid to late adult life and most cases are found in the back of the neck, upper back or shoulders. They are less commonly encountered more anteriorly in the head and neck region. Clinically, they present as painless, slowly growing, superficial lesions indistinguishable from ordinary lipoma.

Histologically, spindle cell lipoma is characterized by an admixture of adipocytes and spindle cells in variable proportions; adipocytes may occasionnally be absent. Spindle cells, arranged in short fascicles, have bland, uniform nuclei and pale eosinophilic “bipolar” cytoplasm. Some nuclear palissadism may be observed. Cells are positive for CD34. The stroma is characterized by the presence of brightly eosinophilic collagen fibres and of numerous mast cells. Some cases show prominent myxoid changes.

In pleomorphic lipoma, mature adipocytes are associated with bizarre, often multinucleated cells (“floret cells ”) and with a few lipoblasts. Interestingly, spindle cell and pleomorphic lipoma share common karyotypic abnormalities (rearrangements of 13q and 16q) and most tumors show hybrid features between both of these lesions.

Spindle cell lipoma must be differentiated from schwannoma and neurofibroma (negativity of spindle cells for S100). Clinical features are primordial in the differential diagnosis between atypical lipoma and pleomorphic lipoma. The relative regularity of adipocytes and the presence of “ropy” collagen also favor the latter. However, as there is some morphological overlap, tumors showing histological features of spindle cell/pleomorphic lipoma but occuring in deeper tissues or in unusual sites should be regarded with diagnostic suspicion. Pleomorphic liposarcoma appears frankly sarcomatous and usually contains “MFH”-like areas. Practically, spindle cell/pleomorphic lipoma should always be considered in the differential diagnosis of lipomatous, spindle cell or myxoid lesions occuring in the upper back/back of the neck area. The rare intradermal spindle cell lipoma have ill-defined margins and are more commonly locate in the face.

Adipose tissue is often present in intramuscular hemangioma. When this fatty component is particularly abundant, it may be mistaken for intramuscular lipoma. However, these lesions should be distinguished as intramuscular angioma tend to recur more often (30-50% of cases) than intramuscular lipoma (20% of cases). Clinically, the majority of intramuscular hemangiomas occur in adolescents and young adults, with a predilection for the head and neck musculature.

The term angiomatosis is used when adjacent tissues such as skin or bone are involved. Angiomyolipoma typically occurs in the kidney, where it is the most common mesenchymal neoplasm, but occasional cases may be predominantly located in the retropenitoneum. Angiomyolipoma is classically composed of an admixture of mature adipose tissue, smooth muscle and thick-walled vessels. It belongs (as lymphangioleiomyomatosis and “sugar tumor” of the lung) to the spectrum of tumors composed of “perivascular epithelioid cells”, characterized immunohistochemically by HMB45 positivity.

Myelolipoma is a rare lesion usually involving the adrenal glands, although occasional cases have been reported in the pelvis and retroperitoneum. The tumor is composed of an admixture of mature adipocytes and hematopoietic elements. There is no association with any hematological disorder.

Liposarcoma is probably the most common soft tissue sarcoma, accounting for approximately 20% of sarcomas. Most patients are adults, with a peak incidence of between 40 to 60 years. Tumors arise predominantly in the lower limbs (35-40%), retroperitoneum (15-20%) and the trunk (20%).

The lipoma-like and sclerosing variants are by far the most common and account together for 40 to 50% of liposarcomas. A transition between areas corresponding to different subtypes is commonly observed.

“Pure” well-differentiated liposarcoma is not capable of metastasis. However, retroperitoneal lesions are rarely completely excised and are associated with significant morbidity and mortality.

Sclerosing liposarcoma is composed of collagenous fibrous tissue, containing scattered adipocytes and atypical stromal cells, which are often multinucleated. Lipoblasts are often hard to find.

Although several forms of lipoma and liposarcoma cells have been described, the present invention is not limited to such cells. Methods and treatment embodiments of the present invention may be directed toward any lipomatous cell or tissue.

One embodiment of the present invention relates to the treatment of lipomatous tumors in humans and companion animals. The benign form of lipomatous tumors is referred to as lipoma, while the rare malignant variant is termed liposarcoma. The various method and treatment embodiments of the present invention are suitable in connection with both the benign and malignant variants. Lipomas present as a soft, well defined mobile mass that grows beneath the skin. Lipomas are a common tumor in dogs and other companion animals, as well as humans.

One embodiment to the present invention is a method of treating lipomatous tumors in mammals comprising administering a therapeutic amount of an antibody directly to tumor cells derived from adipose tissue of a mammal. Such treatments kill cancerous cells, prevent new growth of cancerous cells and prevent regrowth of cancerous cells. Therefore, suitable treatments of the present invention may reduce the size of an lesion of lipomatous cells, inhibit the growth of lipomatous tumor cells, and prevent the regrowth of a lesion of lipomatous cells after their removal.

One embodiment of the present invention is a method of preventing the regrowth of lipomatous tumors after their removal from a subject. Suitable removal techniques involve surgical excision, liposuction, radiation, and any known treatments of lipomatous tumors. One removal technique is described in a reference by Hallock, entitled “Endoscope-assisted suction extraction of lipomas”, described in Annals of Plastic Surgery. 34(1):32-4, January 1995, herein incorporated by reference in its entirety. Another removal technique is described in a reference by Pinski, et al., entitled, “Liposuction of lipomas”, described in Dermatologic Clinics. 8(3):483-92, 1990, herein incorporated by reference in its entirety. The standard therapy for malignant tumors involves surgical excision often combined with radiation therapy. In traditional therapies, local recurrence rates are high after surgical excision. An antibody directed against surface antigens of adipose derived lipomatous tumors, such as sarcomatous tumors may be useful in reducing local recurrence after excision. Such a method would comprise removing the lesion in a subject by any of the described techniques, and subsequently administering an antibody locally to the tissue surrounding the removal site in the subject.

Another embodiment of the present invention is a method of treating lipomatous tumor cells comprising administering a therapeutic amount of an antibody directly to tumor cells of a subject. Another embodiment of the present invention is a composition for the treatment of lipomatous tumors comprising therapeutic antibodies directed against adipocytes.

Antibodies have been generated and found to be effective against normal adipose tissue. However, antibody treatments directed to lipomatous cells have not be disclosed or described. It has been discovered that lipomatous tumor cells and normal human adipose cells share many common surface antigens. Therefore, an antibody specific to the adipocyte cell membrane will also bind to a lipomatous tumor cell derived from adipocytes. Therefore any antibody designed to bind to adipose cells will also be effective in targeting lipomatous cells.

While not wishing to be bound by theory, the lipomatous tumor cells and normal human adipose cells comprise the same cell membranes, including the same receptor and surface antigens. Lipomatous cells are mature adipocyte cells. These atypical adipocytes found in tumors may be varied in size and shape and may have enlarged atypical, nuclei, but the plasma membrane remains unchanged. Therefore, antibodies which are directed against fat cell membranes, will also bind to the surface membrane of lipomatous cells. Therefore, any antibody targeted toward normal adipose cells are suitable in the embodiments of the invention.

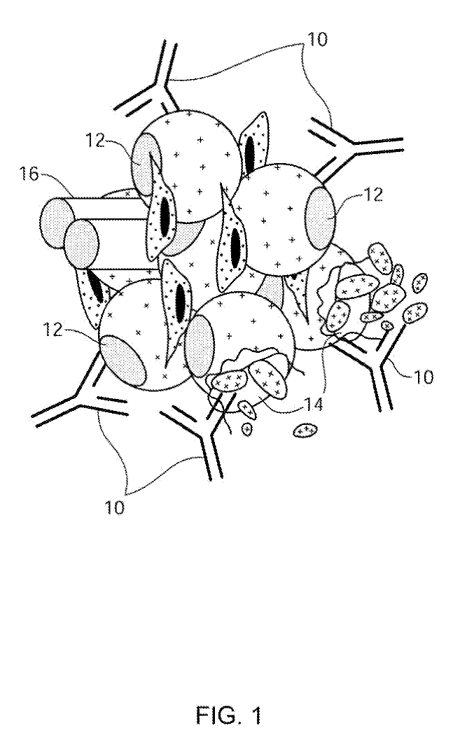

Please refer to FIG. 1 which describes the mechanism of various treatments and methods of the present invention. An antibody directed against fat cells is injected into a tumor and binds only to adipose derived tumor cells. Destruction of bound cells occurs through complement fixation. Other cell types and structure surrounding the lipomatous cells, including blood vessels, nerves and ligaments are not affected. Complement fixation is a known immune response in which an antigen-antibody combination inactivates a complement (so it is unavailable to participate in a second antigen-antibody combination).

An antibody targeted toward normal adipose cells are suitable in the embodiments of the invention. Antibodies directed against adipocytes may be raised by immunization of a serum donor animal with digested adipocyte plasma membranes. For example, a reference authored by Kestin, et al., entitled “Decreased fat content and increased lean in pigs treated with antibodies to adipocyte plasma membranes”, described in Journal of Animal Science, 71(6):1486-94, 1993, is herein incorporated by reference in its entirety. This reference uses antibodies prepared in sheep by an immunization method. This reference also describes the use of the sheep antibodies for use in targeting adipocytes in pigs. This reference reported that upon injection of sheep anti-pig adipocyte serum into the subcutaneous tissues of swine, complete loss of adipose tissue occurred at the site.

Another suitable method for deriving antibodies useful in the present methods and composition embodiments is described in an article by Flint, et al., entitled “Stimulatory and cytotoxic effects of an antiserum to adipocyte plasma membranes on adipose tissue metabolism in vitro and in vivo,” described in Int. J. Obesity 10:69, herein incorporated by reference in its entirety. This reference also describes suitable antibodies useful in the present embodiments including a sheep anti-pig adipocyte plasma membranes.

Another suitable method for deriving antibodies useful in the present methods and composition embodiments is described in U.S. Pat. No. 5,102,658, herein incorporated by reference. This patent describes a fat cell specific antibody which is administered to white fat cell tissues of an animal, or to an antigenic determinant present in the plasma membranes of the white fat cells. Another suitable method for deriving antibodies useful in the present methods and composition embodiments is described in U.S. Pat. No. 5,096,706, herein incorporated by reference. This patent describes a fat cell-specific immunogen which is an antigenic substance selected from plasma membranes of white fat cells.

Suitable antibodies of the present invention may be in the form of antiserum or may be affinity purified by chromatography. Antiserum is a serum that contains antibody or antibodies; it may be obtained from an animal that has been immunized either by injection of antigen into the body or by infection with microorganisms containing the antigen. Antisera may be monovalent (specific for one antigen) or polyvalent (specific for more than one antigen). Commercially available antibodies derived from adipose cells include various Rabbit anti-mouse (and anti-human) Acrp30 (adipocyte complement-related protein of 30 kDa) polyclonal Antibodies, available from Alpha Diagnostic International, Inc. Additional suitable antibodies of the present embodiments include: (1) Rabbit anti-aquaporin adipose specific, polyclonal antibody, available from Chemicon; (2) Chicken anti-aquaporin-O adipose specific, cytoplasmic polyclonal antibody, available from Chemicon; (3) Rabbit anti-human aquaporin, adipose specific polyclonal antibody, available from United States Biological; and (4) Rabbit anti-human resistin, polyclonal antibody, available from United States Biological.

Adipocyte antibodies may also be produced from cultured hybridomas of myeloma cells and lymphocytes secreting the antibodies or through phage display technology. Both methods allow for inexpensive large scale production of antibodies, but the latter method has the advantage of rapidly developing antibodies with a high specificity for target antigens. Moreover, human antibodies may be produced for use in human patients, thereby reducing the risk of allergic reaction.

Antibodies identified by Edwards, et al. in “Isolation and tissue profiles of a large panel of phage antibodies binding to the human adipocyte cell surface”, Journal of Immunological Methods. 245(1-2):67-78, 2000, herein incorporated by reference, are suitable in the methods and compositions of the present invention. This reference identified anti-adipocyte antibodies such as FAT.13, which binds to adipocytes in the tissue panel of 37 different human tissues. This reference surveyed the surface antigens of the human adipocyte cell.

Using phage display technology, antibodies have been produced that bind to human adipocytes and cross react only with capillaries when screened against a panel of 37 different human tissues. Therefore, present treatments and methods of the present invention will be effective against lipomatous cells, but will not adversely affect other non-cancerous cells typically found within adipose tumors. These may include blood vessels, nerves and ligaments. These antibodies are suitable in the present embodiments and are described in Dickinson, et al., “Antibody-induced lysis of isolated rat epididymal adipocytes and complement activation in vivo”, Obesity Research, 10(2):122-7, 2002, herein incorporated by reference. These antibodies recognize antigens on the surface of adipocytes across different species. The mechanism of adipocyte cell destruction is mediated by complement. When such antibodies are administered systemically into rats, serum complement levels decrease.

Antibodies suitable for the present invention include antibodies derived from mammals, specifically humans and companion animals such as dogs. Antibodies may be raised from an appropriate source known as a donor or donor mammal. Additionally, in the method and composition embodiments, the antibodies derived from any suitable source may be administered or injected into a mammal, specifically humans and companion animals such as dogs. The source of the antibodies and the subject of a treatment does not need to be the same specific mammal. For example, a human subject may be administered an antibody raised in a companion animal. Additionally, all other combinations are possible. It is preferred that a human antibody is administered to a human subject, however to minimize the risk of allergic reaction.

One embodiment of the invention is the treatment of benign subcutaneous fatty tumors in humans and companion animals, such as dogs, by direct intralesional injection of antibodies. Additionally, this invention applies to the treatment of malignant adipose derived tumors, in which case the antibodies are injected into the lesion and into surrounding tissues to minimize local recurrence.

“Providing” when used in conjunction with a therapeutic means to administer a therapeutic directly into or onto a target tissue or to administer a therapeutic to a patient whereby the therapeutic positively impacts the tissue to which it is targeted. Thus, as used herein, the term “providing”, when used in conjunction with compositions comprising therapeutic antibodies may include, but is not limited to, providing compositions into or onto the target tissue; providing compositions systemically to a patient by, e.g., intravenous injection whereby the therapeutic reaches the target tissue; providing an compositions in the form of the encoding sequence thereof to the target tissue (e.g., by so-called gene-therapy techniques).

Intralesional injection is a known method. It may be accomplished with corticosteroid suspensions. Intralesional injection of a corticosteroid suspension (almost always triamcinolone acetonide) is useful for delivering a high concentration of drug to a chronic lesion or to one resistant to topical corticosteroids. The suspension may be diluted with sterile saline and is usually used at concentrations of 2.5 to 5 mg/mL to minimize risk of local dermal atrophy and, in black patients, hypopigmentation. Dermal atrophy is usually reversible. Higher concentrations of up to 40 mg/mL may also be used.

As used herein, the term “therapeutic” means an composition utilized to treat, combat, ameliorate, prevent or improve an unwanted condition or disease of a patient. In part, embodiments of the present invention are directed to eliminate, prevent the regrowth, and inhibit proliferation of lipomatous cells. Therefore, embodiments of the invention that provide for a “therapeutic amount”, a “therapeutically effective amount” or a “therapeutic treatment”, are compositions that effectuate the elimination, prevention, regrowth, or inhibition of lipomatous cells.

All of the compositions and methods disclosed and claimed herein may be made and executed without undue experimentation in light of the present disclosure. While the compositions and methods of this invention have been described in terms of preferred embodiments, it will be apparent to those of skill in the art that variations may be applied to the composition, methods, and in the steps or in the sequence of steps of the method described herein, without departing from the concept, spirit and scope of the invention.

What is claimed is:

1. A method of treating lipomatous tumor cells in a mammal subject comprising administering to tumor cells a therapeutic amount of an antibody derived from adipose tissue of a mammal donor.

2. The method according to claim 1, wherein the administration is by intralesional injection directed against the tumor cells.

3. The method according to claim 1, wherein the mammal subject is a human.

4. The method according to claim 1, wherein the mammal subject is a companion animal.

5. The method according to claim 1, wherein the subject mammal and donor mammal are human.

6. The method according to claim 1, wherein the tumor cells are a lyposaroma.

7. The method according to claim 1, wherein the tumor cells are a lipoma.

8. A method of eliminating lipomatous tumor cells in a mammal subject comprising administering to the tumor cells a therapeutic amount of an antibody derived from adipose tissue of a mammal donor.

9. The method according to claim 8, wherein the administration is by intralesional injection directed against the tumor cells.

10. The method according to claim 8, wherein the mammal subject is a human.

11. The method according to claim 8, wherein the mammal subject is a companion animal.

12. The method according to claim 8, wherein the tumor cells are a lyposaroma.

13. The method according to claim 8, wherein the tumor cells are a lipoma.

14. A method of inhibiting proliferation of lipomatous tumor cells in a mammal subject comprising administering to the tumor cells a therapeutic amount of an antibody derived from adipose tissue of a mammal donor.

15. The method according to claim 14, wherein the administration is by intralesional injection.

16. The method according to claim 14, wherein the mammal subject is a human.

17. The method according to claim 14, wherein the mammal subject is a companion animal.

18. The method according to claim 14, wherein the tumor cells are a liposarcoma.

19. The method according to claim 14, wherein the tumor cells are a lipoma.

20. A method of preventing the regrowth of lipomatous tumor cells in a mammal subject comprising:
removing a lesion of lipomatous cells by surgical procedure; and
administering a therapeutic amount of an antibody to tissue surrounding the removed lesion, wherein the antibody is derived from adipose tissue of a mammal donor.

21. The method according to claim 20, wherein the administration is by intralesional injection.

22. The method according to claim 20, wherein the mammal subject is a human.

23. The method according to claim 20, wherein the mammal subject is a companion animal.

24. The method according to claim 20, wherein the lesion is a liposarcoma.

25. The method according to claim 20, wherein the lesion is a lipoma.

26. A therapeutic composition for the treatment of lipomatous tumors comprising a therapeutic amount of an antibody directed against adipocytes.
